(19) United States
(12) Patent Application Publication
(10) Pub. No.: US 2013/0150611 A1
(43) Pub. Date: Jun. 13, 2013

(54) PROCESS FOR PRODUCING CARBOXYLIC ACID ESTER

(71) Applicant: Koji Hagiya, Osaka-shi (JP)

(72) Inventor: Koji Hagiya, Osaka-shi (JP)

(73) Assignee: SUMITOMO CHEMICAL COMPANY, LIMITED, Chuo-ku, Tokyo (JP)

(21) Appl. No.: 13/817,352

(22) PCT Filed: Aug. 25, 2011
(86) PCT No.: PCT/JP2011/069858

§ 371 Date: Feb. 15, 2013

(30) Foreign Application Priority Data
Aug. 26, 2010 (JP) 2010-189175
Jul. 7, 2011 (JP) 2011-150781

(51) Int. Cl. C07C 67/44

(52) U.S. Cl. CPC C07C 67/44
USPC 560/51; 560/152; 560/174; 560/238

(57) ABSTRACT

The present invention relates to a process for producing a carboxylic acid ester, comprising a step of oxidizing an aldehyde by mixing an alcohol, carbon dioxide, the aldehyde and at least one compound selected from the group consisting of compounds represented by the formulae (2-1) and (2-2):

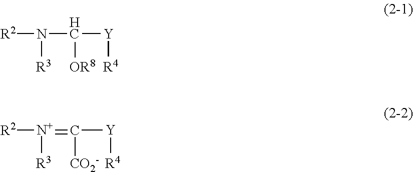

wherein R² represents an alkyl group optionally having a substituent or the like; R³ and R⁴ each independently represents an alkyl group optionally having a substituent or the like or R³ and R⁴ are linked together to form a divalent hydrocarbon group optionally having a substituent or the like; Y represents a group of —S— or a group of —N(R⁵)—, wherein R⁵ represents an alkyl group optionally having a substituent or the like, or R⁵ is linked to R⁴ to form a divalent hydrocarbon group optionally having a substituent; and R⁸ represents an alkyl group.

### TECHNICAL FIELD

The present application is filed, claiming the priorities based on the Japanese Patent Application Nos. 2010-189175 (filed on Aug. 26, 2010) and 2011-150781 (filed on Jul. 7, 2011), and a whole of the contents of these applications is incorporated herein by reference.

The present invention relates to a process for producing a carboxylic acid ester.

### BACKGROUND ART

Carboxylic acid esters are important as various chemical products. Among them, ketocarboxylic acid esters are known to be useful as intermediates in the preparation of amino acids and the like.

Heretofore, some processes for producing carboxylic acid esters from aldehydes have been developed. For example, Non-patent literature 1 (see, e.g., Table 1) discloses a process for producing methyl 4-nitrobenzoate as a carboxylic acid ester by reacting 4-nitrobenzaldehyde as an aldehyde, an alcohol, iodine and sodium nitrite. Non-patent literature 2 (see, e.g., Supporting information, page 2) discloses a process for producing cinnamic acid ester as a carboxylic acid ester by reacting cinnamaldehyde as an aldehyde and a quinone in the presence of a catalyst prepared by a triazolium salt and 1,8-diazabicyclo[5.4.0]-undeca-7-ene.

Non-patent literature 1: Synthesis, pages 276 to 282 (2010)
Non-patent literature 2: Journal of the American Chemical Society, Vol. 132, pages 1190 to 1191 (2010)

### DISCLOSURE OF INVENTION

### Problem to be Solved by the Invention

An object of the present invention is to provide a novel process for producing a carboxylic acid ester.

### Means for Solving the Problem

As a result of the present inventors' intensive studies for solving the above-described problem, the present invention is accomplished.

The present invention provides the followings:

[1] A process for producing a carboxylic acid ester, comprising a step of oxidizing an aldehyde by mixing an alcohol, carbon dioxide, the aldehyde and at least one compound selected from the group consisting of a compound represented by the formula (2-1):

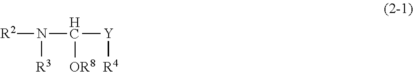

wherein R² represents an alkyl group optionally having a substituent or an aryl group optionally having a substituent;
R³ and R⁴ each independently represents an alkyl group optionally having a substituent or an aryl group optionally having a substituent, or R³ and R⁴ are linked together to form a divalent hydrocarbon group optionally having a substituent or a group of —CH=N— optionally having a substituent;
Y represents a group of —S— or a group of —N(R⁵)—, wherein R⁵ represents an alkyl group optionally having a substituent or an aryl group optionally having a substituent, or R⁵ is linked to R⁴ to form a divalent hydrocarbon group optionally having a substituent; and
R⁸ represents an alkyl group; and
a compound represented by the formula (2-2):

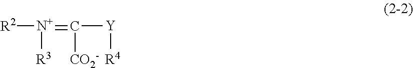

wherein R², R³, R⁴ and Y are as defined above.
[2] The process according to the above item [1], wherein the process is carried out in the presence of oxygen.
[3] The process according to the above item [1] or [2], wherein the aldehyde is a compound represented by the formula (1):

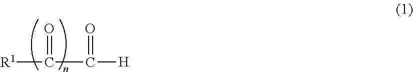

wherein R⁴ represents a hydrocarbon group optionally having a substituent or a heteroaryl group optionally having a substituent, and
n represents 0 or 1;
the alcohol is a compound represented by the formula (4):

R⁹—OH (4)

wherein R⁹ represents an alkyl group optionally having a substituent; and
the carboxylic acid ester is a compound represented by the formula (3):

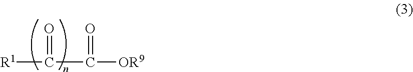

wherein R′, R⁹ and n are as defined above.
[4] The process according to the above item [1], [2] or [3], wherein the compound represented by the formula (2-1) is a compound represented by the formula (2-3):

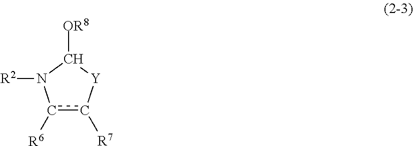

wherein R², R⁸ and Y are as defined above;
R⁶ and R⁷ each independently represents a hydrogen atom, an alkyl group optionally having a substituent or an aryl group optionally having a substituent, or R⁶ and R⁷ are taken together with the carbon atoms to which they are attached to form a ring; and [symbol]
represents a single bound or a double bond; or a compound represented by the formula (2-4):

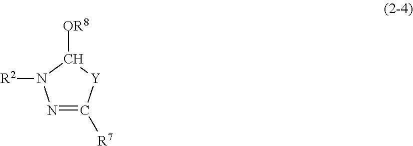

wherein R², R⁷, R⁸ and Y are as defined above; and the compound represented by the formula (2-2) is a compound represented by the formula (2-5):

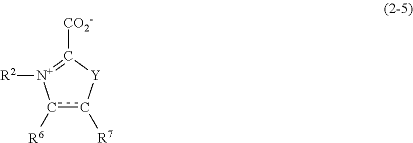

wherein R², R⁶, R⁷ and Y are as defined above; or a compound represented by the formula (2-6):

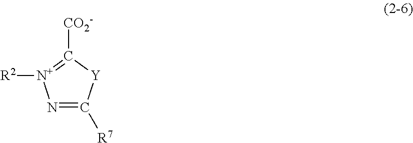

wherein R², R⁷ and Y are as defined above.
[5] The process according to the above item [4], wherein in the formulae (2-3), (2-4), (2-5) and (2-6), Y is a group of —N(R⁵)—; and R² and R⁵ each is independently a phenyl group optionally having a substituent.
[6] A process for producing a carboxylic acid ester, comprising a step of reacting an aldehyde, an alcohol and carbon dioxide in the presence of at least one compound selected from the group consisting of a compound represented by the formula (2-1):

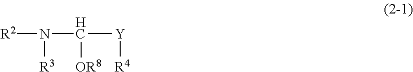

wherein R² represents an alkyl group optionally having a substituent or an aryl group optionally having a substituent; R³ and R⁴ each independently represents an alkyl group optionally having a substituent or an aryl group optionally having a substituent, or R³ and R⁴ are linked together to form a divalent hydrocarbon group optionally having a substituent or a group of —CH═N— optionally having a substituent;
Y represents a group of —S— or a group of —N(R⁵)—, wherein R⁵ represents an alkyl group optionally having a substituent or an aryl group optionally having a substituent, or R⁵ is linked to R⁴ to form a divalent hydrocarbon group optionally having a substituent; and
R⁸ represents an alkyl group; and
a compound represented by the formula (2-2):

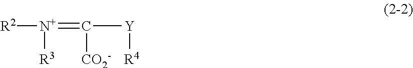

wherein R², R³, R⁴ and Y are as defined above.
[7] The process according to the above item [6], wherein the process is carried out in the presence of oxygen.
[8] The process according to the above item [6] or [7], wherein the aldehyde is a compound represented by the formula (1):

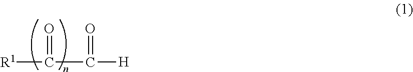

wherein R¹ represents a hydrocarbon group optionally having a substituent or a heteroaryl group optionally having a substituent; and
n represents 0 or 1;
the alcohol is a compound represented by the formula (4):

R⁹—OH (4)

wherein R⁹ represents an alkyl group optionally having a substituent; and
the carboxylic acid ester is a compound represented by the formula (3):

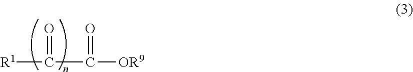

wherein R¹, R⁹ and n are as defined above.
[9] The process according to the above item [6], [7] or [8], wherein the compound represented by the formula (2-1) is a compound represented by the formula (2-3):

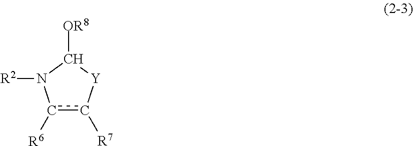

wherein R², R⁸ and Y are as defined above;
R⁶ and R⁷ each independently represents a hydrogen atom, an alkyl group optionally having a substituent or an aryl group optionally having a substituent, or R⁶ and R⁷ are taken together with the carbon atoms to which they are attached to form a ring; and [symbol]
represents a single bound or a double bond; or a compound represented by the formula (2-4):

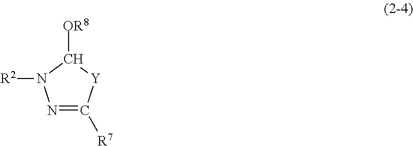

wherein R², R⁷, R⁸ and Y are as defined above; and the compound represented by the formula (2-2) is a compound represented by the formula (2-5):

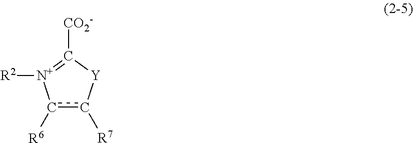

wherein R², R⁶, R⁷ and Y are as defined above; or a compound represented by the formula (2-6):

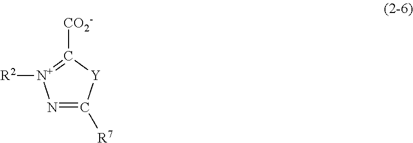

wherein R², R⁷ and Y are as defined above.

[10] The process according to the above item [9], wherein in the formulae (2-3), (2-4), (2-5) and (2-6), Y is a group of —N(R⁵)—; and R² and R⁵ each is independently a phenyl group optionally having a substituent.

According to the present invention, a novel process for producing a carboxylic acid ester from an aldehyde can be provided.

### MODES FOR CARRYING OUT THE INVENTION

The process for a producing a carboxylic acid ester according to the present invention is characterized by comprising a step of oxidizing an aldehyde by mixing an alcohol, carbon dioxide, the aldehyde and at least one compound selected from the group consisting of a compound represented by the formula (2-1) (hereinafter sometimes referred to as “compound (2-1)”) and a compound represented by the formula (2-2) (hereinafter sometimes referred to as “compound (2-2)”), or by comprising a step of reacting an aldehyde, an alcohol and carbon dioxide in the presence of at least one compound selected from the group consisting of the compounds (2-1) and (2-2).

Hereinafter, the oxidation of an aldehyde is sometimes referred to as the “present reaction”. Also, the reaction of an aldehyde, an alcohol and carbon dioxide is sometimes referred to as the “present reaction”. In the present reaction, an aldehyde is oxidized and converted to a carboxylic acid ester.

Firstly, the aldehyde to be used in the present invention will be described below.

In the present invention, any aldehyde may be used as long as it is a compound having a group of “—CHO”, but preferred examples thereof include those represented by the formula (1) (hereinafter sometimes referred to as “compound (1)”).

Hereinafter, the present invention will be described in detail with reference to embodiments using the compound (1) as an aldehyde.

In the formula (1), examples of the “hydrocarbon group optionally having a substituent” for R¹ include an alkyl group optionally having a substituent, an alkenyl group optionally having a substituent and an aryl group optionally having a substituent.

As to R¹, examples of the alkyl group in the “alkyl group optionally having a substituent” include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and cyclic C₃-C₁₂ alkyl groups such as a cyclopropyl group, 2,2-dimethylcyclopropyl group, a cyclopentyl group, a cyclohexyl group and a menthyl group.

Examples of the substituent which the alkyl group may have include a group selected from the following Group G1:

### <Group G1>

a C₁-C₁₀ alkoxy group optionally having a fluorine atom;
a C₇-C₂₀ aralkyloxy group optionally having a C₁-C₁₀ alkoxy group;
a C₇-C₂₀ aralkyloxy group having a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group;
a C₂-C₁₀ acyl group optionally having a C₁-C₁₀ alkoxy group;
a C₁-C₁₀ alkylthio group;
a C₂-C₁₀ alkoxycarbonyl group;
a C₆-C₂₀ aryl group;
a C₅-C₂₀ heteroaryl group; and a halogen atom.

In Group G1, examples of the “C₁-C₁₀ alkoxy group optionally having a fluorine atom” include a methoxy group, an ethoxy group, a propoxy group, an isopropoxy group, a butoxy group, an isobutoxy group, a sec-butoxy group, a tert-butoxy group and a trifluoromethyloxy group.

Examples of the “C₇-C₂₀ aralkyloxy group optionally having a C₁-C₁₀ alkoxy group” include a benzyloxygroup, a 4-methylbenzyloxy group and a 4-methoxybenzyloxy group.

Examples of the “C₇-C₂₀ aralkyloxy group having a C₆-C₁₀ aryloxy group” include a 3-phenoxybenzyloxy group.

Examples of the “C₆-C₁₀ aryloxy group optionally having a C₁-C₁₀ alkoxy group” include a phenoxy group, a 2-methylphenoxy group, a 4-methylphenoxy group and a 4-methoxyphenoxy group.

Examples of the “C₆-C₁₀ aryloxy group having a C₆-C₁₀ aryloxy group” include a 3-phenoxyphenoxy group.

Examples of the “C₂-C₁₀ acyl group optionally having a C₁-C₁₀ alkoxy group” include an acetyl group, a propionyl group, a benzylcarbonyl group, a 4-methylbenzylcarbonyl group, a 4-methoxybenzylcarbonyl group, a benzoyl group, a 2-methylbenzoyl group, 4-methylbenzoyl group and 4-methoxybenzoyl group.

Examples of the “C₁-C₁₀ alkylthio group” include a methylthio group, an ethylthio group and an isopropylthio group.

Examples of the “C₂-C₁₀ alkoxycarbonyl group” include a methoxycarbonyl group and an ethoxycarbonyl group.

Examples of the “C₆-C₂₀ aryl group” include a phenyl group, a 1-naphthyl group and a 2-naphthyl group.

Examples of the “C₅-C₂₀ heteroaryl group” include a 2-pyridyl group, a 3-pyridyl group, a 4-pyridyl group, a 2-quinolyl group, a 3-quinolyl group and a 4-quinolyl group.

Examples of the “halogen atom” include a fluorine atom, a chlorine atom and a bromine atom.

Examples of the alkyl group having a group selected from Group G1 include a chloromethyl group, a fluoromethyl group, a trifluoromethyl group, a methoxymethyl group, an ethoxymethyl group, a 1-methoxyethyl group, a 2-methoxyethyl group, a methoxycarbonylmethyl group, a phenylmethyl group, a 2-pyridylmethyl group, a 3-pyridylmethyl group, a 1-ethoxycarbonyl-2,2-dimethyl-3-cyclopropyl group and a 2-methylthioethyl group.

As to R¹, examples of the alkenyl group in the “alkenyl group optionally having a substituent” include linear, branched or cyclic C₂-C₁₂ alkenyl groups such as a vinyl group, a 1-propenyl group, a 1-butenyl group, a 2-methyl-1-propenyl group, and a 1-cyclohexenyl group.

Examples of the substituent which the alkenyl group may have include a group selected from the above Group G1.

Examples of the alkenyl group having a group selected from Group G1 include a 2-chlorovinyl group and a 2-trifluoromethylvinyl group.

As to R¹, examples of the aryl group in the “aryl group optionally having a substituent” include C₆-C₂₀ aryl groups such as a phenyl group, a 2-methylphenyl group, a 4-methylphenyl group, a 1-naphthyl group, a 2-naphthyl group and a styryl group.

Examples of the substituent which the aryl group may have include a group selected from the following Group G2:

### <Group G2>

a C₁-C₁₀ alkoxy group optionally having a fluorine atom or
a C₁-C₁₀ alkoxy group;
a C₆-C₁₀ aryloxy group optionally having a C₁-C₁₀ alkoxy group;
a C₆-C₁₀ aryloxy group having a C₆-C₁₀ aryloxy group;
a C₂-C₁₀ acyl group optionally having a C₁-C₁₀ alkoxy group;
a C₁-C₆ alkylenedioxy group;
a nitro group; and
a halogen atom.

In Group G2, examples of the “C₁-C₁₀ alkoxy group optionally having a fluorine atom or a C₁-C₁₀ alkoxy group” include a methoxy group, an ethoxy group, a propoxy group, an isopropoxy group, a butoxy group, an isobutoxy group, a sec-butoxy group, a tert-butoxy group, a pentyloxy group, a cyclopentyloxy group, a fluoromethoxy group, a trifluoromethoxy group, a methoxymethoxy group, an ethoxymethoxy group and a methoxyethoxy group.

Examples of the “C₆-C₁₀ aryloxy group optionally having a C₁-C₁₀ alkoxy group” include a phenoxy group, a 2-methylphenoxy group, a 4-methylphenoxy group and a 4-methoxyphenoxy group.

Examples of the “C₆-C₁₀ aryloxy group having a C₆-C₁₀ aryloxy group” include a 3-phenoxyphenoxy group.

Examples of the “C₂-C₁₀ acyl group optionally having a C₁-C₁₀ alkoxy group” include an acetyl group, a propionyl group, a benzylcarbonyl group, a 4-methylbenzylcarbonyl group and a 4-methoxybenzylcarbonyl group.

Examples of the “C₁-C₆ alkylenedioxy group” include a methylenedioxy group and an ethylenedioxy group.

Examples of the “halogen atom” include a fluorine atom and a chlorine atom.

Examples of the aryl group having a group selected from Group G2 include a 4-chlorophenyl group, a 4-methoxyphenyl group and a 3-phenoxyphenyl group.

As to R¹, the heteroaryl group in the “heteroaryl group optionally having a substituent” include C₄-C₁₀ heteroaryl groups having at least one hetero atom such as a nitrogen atom, an oxygen atom and a sulfur atom. Specific examples of the heteroaryl group include a 2-pyridyl group, a 3-pyridyl, a 4-pyridyl, a 2-furyl group, a 3-furyl group, a 5-methyl-2-furyl group and a 2-chloro-3-pyridinyl group.

Examples of the compound (1) wherein n represents 0 in the formula (1) include benzaldehyde, 4-chlorobenzaldehyde, 2-methylbenzaldehyde, 4-fluorobenzaldehyde, 2-methoxybenzaldehyde, 2,4-dichlorobenzaldehyde, 2-nitrobenzaldehyde, 2-naphthylbenzaldehyde, 2-pyridylaldehyde, acetaldehyde, n-propylaldehyde, isopropylaldehyde, n-butylaldehyde, n-pentylaldehyde, n-hexylaldehyde, n-heptylaldehyde, n-octylaldehyde, cyclohexylaldehyde, 4-(methylthio)-1-butanal, acrolein and 3-phenyl-2-propenealdehyde.

The compound (1) wherein n represents 0 in the formula (1) may be a commercially available product or one prepared by any known method such as a method comprising oxidation of an alkyl alcohol or benzenemethanol, a method comprising hydroformylation of a terminal double bond, or a method comprising dihalogenation of a methyl group and subsequent hydrolysis.

Examples of the compound (1) wherein n represents 1 in the formula (1) include phenylglyoxal, 4-chlorophenylglyoxal, 2-methyl phenylglyoxal, 4-fluorophenylglyoxal, 2-methoxyphenylglyoxal, 2,4-dichlorophenylglyoxal, 2-nitrophenylglyoxal, 2-naphthylglyoxal, 2-pyridineglyoxylaldehyde, methylglyoxal, ethylglyoxal, n-propylglyoxal, isopropylglyoxal, cyclohexylglyoxal, 4-(methylthio)-2-oxo-1-butanal, vinylglyoxal and styrylglyoxal.

The compound (1) wherein n represents 1 in the formula (1) may be a commercially available product or one prepared by any known method such as a method comprising oxidation of a ketoalcohol with oxygen in the presense of a metal catalyst (see, e.g., JP-A-2000-336055).

Next, the alcohol to be used in the present invention will be described.

In the present invention, the alcohol to be used is not limited, but preferable examples thereof include a compound represented by the formula (4) (hereinafter sometimes referred to as “compound (4)”).

In the formula (4), examples of the alkyl group in the “alkyl group optionally having a substituent” for R⁹ include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and cyclic C₃-C₁₂ alkyl groups such as a cyclopropyl group, a 2,2-dimethylcyclopropyl group, a cyclopentyl group, a cyclohexyl group and a menthyl group.

Examples of the substituent which the alkyl group may have include a group selected from the following Group G3:

### <Group G3>

a C₁-C₁₀ alkoxy group optionally having a fluorine atom;
a C₇-C₂₀ aralkyloxy group optionally having a C₁-C₁₀ alkoxy group;
a C₇-C₂₀ aralkyloxy group having a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group;
a C_{2—}C₁₀ acyl group optionally having a C₁-C₁₀ alkoxy group;
a C₁-C₁₀ alkylthio group;
a C_{2—}C₁₀ alkoxycarbonyl group;
a C₆-C₂₀ aryl group;
a C_{5—}C₂₀ heteroaryl group; and a halogen atom.

In the Group G3, examples of the “C₁-C₁₀ alkoxy group optionally having a fluorine atom” include a methoxy group, an ethoxy group, a propoxy group, an isopropoxy group, a butoxy group, an isobutoxy group, a sec-butoxy group, a tert-butoxy group and a trifluoromethyloxy group.

Examples of the “C₇-C₂₀ aralkyloxy group optionally having a C₁-C₁₀ alkoxy group” include a benzyloxy group, a 4-methylbenzyloxy group and a 4-methoxybenzyloxy group.

Examples of the “C₇-C₂₀ aralkyloxy group having a C₆-C₁₀ aryloxy group” include a 3-phenoxybenzyloxy group.

Examples of the “C₆-C₁₀ aryloxy group optionally having a C₁-C₁₀ alkoxy group” include a phenoxy group, a 2-methylphenoxy group, a 4-methylphenoxy group and a 4-methoxyphenoxy group.

Examples of the “C₆-C₁₀ aryloxy group having a C₆⁻C₁₀ aryloxy group” include a 3-phenoxyphenoxy group. Examples of the “C₂-C₁₀ acyl group optionally having a C₁-C₁₀ alkoxy group” include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group and a 4-methoxybenzoyl group.

Examples of the “C₁-C₁₀ alkylthio group” include a methylthio group, an ethylthio group and an isopropylthio group.

Examples of the “C₂-C₁₀ alkoxycarbonyl group” include a methoxycarbonyl group and an ethoxycarbonyl group.

Examples of the “C₆-C₂₀ aryl group” include a phenyl group, a 1-naphthyl group and a 2-naphthyl group.

Examples of the “C₅-C₂₀ heteroaryl group” include a 2-pyridyl group, a 3-pyridyl group, a 4-pyridyl group, a 2-quinolyl group, a 3-quinolyl group and a 4-quinolyl group.

Examples of the “halogen atom” include a fluorine atom, a chlorine atom and a bromine atom.

Examples of the alkyl group having a group selected from Group G3 include a chloromethyl group, a fluoromethyl group, a trifluoromethyl group, a methoxymethyl group, an ethoxymethyl group, a 1-methoxyethyl group, a 2-methoxyethyl group, a methoxycarbonylmethyl group, a phenylmethyl group, a 2-pyridylmethyl group, a 3-pyridylmethyl group, a 1-ethoxycarbonyl-2,2-dimethyl-3-cyclopropyl group and a 2-methylthioethyl group.

Examples of the compound (4) include methanol, ethanol, 1-propanol, isopropanol, 1-butanol, isobutanol, 1-pentanol, 1-hexanol, cyclohexanol, benzylalcohol, 2-phenylethanol and 2-pyridinemethanol.

The compound (4) may be a commercially available product or one prepared by any known method such as a method comprising partial oxidation of an alkane or an alkyl-substituted benzene, a method comprising addition of water to a double bond, or a fermentation method.

The amount of the alcohol to be used is preferably 1 mol or more relative to 1 mol of an aldehyde. The upper limit of the amount is not limited, but it is preferably 100 mol or less from an economic point of view.

Hereinafter, the compounds (2-1) and (2-2) will be described.

In the formulae (2-1) and (2-2), examples of the alkyl group in the “alkyl group optionally having a substituent” for R³ and R⁴ include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and cyclic C₃-C₁₂ alkyl groups such as a cyclopropyl group, a 2,2-dimethylcyclopropyl group, a cyclopentyl group, a cyclohexyl group and a menthyl group.

Examples of the substituent which the alkyl group may have include a group selected from the following Group G4:

### <Group G4>

a C₆-C₁₀ aryl group optionally having a C₁-C₁₀ alkoxy group;
a C₁-C₁₀ alkoxy group optionally having a fluorine atom;
a benzyloxy group optionally having at least one group selected from the group consisting of a C₁-C₁₀ alkoxy group,
a C₁-C₁₀ alkyl group and a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group;
a C_{2—}C₁₀ acyl group optionally having a C₁-C₁₀ alkoxy group;
a carboxy group;
and
a fluorine atom.

In the Group G4, examples of the “C₆-C₁₀ aryl group optionally having a C₁-C₁₀ alkoxy group” include a phenyl group, a naphthyl group, a 4-methyl phenyl group and a 4-methoxyphenyl group.

Examples of the “C₁-C₁₀ alkoxy group optionally having a fluorine atom” include a methoxy group, an ethoxy group, a propoxy group, an isopropoxy group, a butoxy group, an isobutoxy group, a sec-butoxy group, a tert-butoxy group and a trifluoromethoxy group.

Examples of the “benzyloxy group optionally having at least one group selected from the group consisting of a C₁-C₁₀ alkoxy group, a C₁⁻C₁₀ alkyl group and a C₆-C₁₀ aryloxy group” include a benzyloxy group, 4-methylbenzyloxy group, a 4-methoxybenzyloxy group and a 3-phenoxybenzyloxy group.

Examples of the “C₆-C₁₀ aryloxy group optionally having a C₁-C₁₀ alkoxy group” include a phenoxy group, a 2-methylphenoxy group, a 4-methylphenoxy group and a 4-methoxyphenoxy group.

Examples of the “C₆-C₁₀ aryloxy group having a C₆⁻C₁₀ aryloxy group” include a 3-phenoxyphenoxy group. Examples of the “C₂-C₁₀ acyl group optionally having a C₁-C₁₀ alkoxy group” include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group and a 4-methoxybenzoyl group.

Examples of the alkyl group having a group selected from Group G4 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

In the formulae (2-1) and (2-2), examples of the aryl group in the “aryl group optionally having a substituent” for R³ and R⁴ include C₆-C₁₀ aryl groups such as a phenyl group, a 2-methylphenyl group, a 4-methylphenyl group, a 1-naphthyl group and a 2-naphthyl group.

Examples of the substituent which the aryl group may have include a group selected from the above Group G2.

Examples of the aryl group having a group selected from Group G2 include a 4-chlorophenyl group and a 4-methoxyphenyl group.

In the formulae (2-1) and (2-2), R³ and R⁴ may be linked together to form a divalent hydrocarbon group optionally having a substituent. Examples of the divalent hydrocarbon group include a ethylene group, a trimethylene group, a vinylene group, a propane-1,2-diyl group, a propene-1,2-diyl group, a butane-1,2-diyl group, a 2-butene-1,2-diyl group, a cyclopentane-1,2-diyl group, a cyclohexane-1,2-diyl group, an o-phenylene group, a 1,2-diphenyl ethylene group and a 1,2-diphenylvinylene group.

Examples of the substituent which the divalent hydrocarbon group may have include a group selected from the above Group G2.

As to R³ and R⁴, it is preferred that they are linked together to form a divalent hydrocarbon group optionally having a substituent.

In the formulae (2-1) and (2-1), examples of the alkyl group in the “alkyl group optionally having a substituent” for R² and R⁵ include linear or branched C₁-C₁₂ alkyl groups such as a methyl group, an ethyl group, a propyl group, an isopropyl group, a butyl group, an isobutyl group, a sec-butyl group, a tert-butyl group, a pentyl group, tert- pentyl group and a decyl group; and cyclic C₃⁻C₁₂ alkyl groups such as a cyclopropyl group, a 2,2-dimethylcyclopropyl group, a cyclopentyl group, a cyclohexyl group, a menthyl group and an adamantyl group.

Examples of the substituent which the alkyl group may have include a group selected from the following Group 5:

### <Group G5>

a C₆-C₁₀ aryl group optionally having a C₁-C₁₀ alkoxy group;
a C₁-C₁₀ alkoxy group optionally having a fluorine atom;
a C₇-C₂₀ aralkyloxy group optionally having a C₁-C₁₀ alkoxy group;
a C₇-C₂₀ aralkyloxy group having a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group;
and
a C₂-C₁₀ acyl group optionally having a C₁-C₁₀ alkoxy group.

In the Group G5, examples of the “C₆-C₁₀ aryl group optionally having a C₁-C₁₀ alkoxy group” include a phenyl group, a naphthyl group, a 4-methyl phenyl group and a 4-methoxyphenyl group.

Examples of the “C₁-C₁₀ alkoxy group optionally having a fluorine atom” include a methoxy group, an ethoxy group, a propoxy group, an isopropoxy group, a butoxy group, an isobutoxy group, a sec-butoxy group, a tert-butoxy group and a trifluoromethoxy group.

Examples of the “C₇-C₂₀ aralkyloxy group optionally having a C₁-C₁₀ alkoxy group” include a benzyloxy group, a 4-methylbenzyloxy group and a 4-methoxybenzyloxy group.

Examples of the “C₇-C₂₀ aralkyloxy group having a C_{6-C}₁₀ aryloxy group” include a 3-phenoxybenzyloxy group. Examples of the “C₆-C₁₀ aryloxy group optionally having a C₁-C₁₀ alkoxy group” include a phenoxy group, a 2-methylphenoxy group, a 4-methylphenoxy group and a 4-methoxyphenoxy group.

Examples of the “C₆-C₁₀ aryloxy group having a C₆-C₁₀ aryloxy group” include a 3-phenoxyphenoxy group.

Examples of the C₂-C₁₀ acyl group optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group and a 4-methoxybenzoyl group.

Examples of the alkyl group having a group selected from Group G5 include a methoxymethyl group, an ethoxymethyl group, a 1-methoxyethyl group, a 2-methoxyethyl group, a benzyl group, a 4-fluorobenzyl group, a 4-methylbenzyl group, a phenoxymethyl group, a 2-oxopropyl group, a 2-oxobutyl group and a phenacyl group.

In the formulae (2-1) and (2-2), examples of the aryl group in the “aryl group optionally having a substituent” for R² and R⁵ include C₆-C₂₀ aryl groups such as a phenyl group, a 1-naphthyl group, a 2-naphthyl group, a 2-methylphenyl group, a 4-methylphenyl group, a 2,6-dimethylphenyl group, a 2,4,6-trimethylphenyl group and a 2,6-diisopropylphenyl group.

Examples of the substituent which the aryl group may have include a group selected from the following Group G6:

### <Group G6>

a C₁-C₁₀ alkoxy group optionally having a fluorine atom or
a C₁-C₁₀ alkoxy group; and a halogen atom.

In the Group G6, examples of the “C₁-C₁₀ alkoxy group optionally having a fluorine atom or a C₁-C₁₀ alkoxy group” include a methoxy group, an ethoxy group, a propoxy group, an isopropoxy group, a butoxy group, an isobutoxy group, a sec-butoxy group, a tert-butoxy group, a pentyloxy group, a cyclopentyloxy group, a fluoromethoxy group, a trifluoromethoxy group, a methoxymethoxy group, an ethoxymethoxy group and a methoxyethoxy group.

Examples of the “halogen atom” include a fluorine atom and a chlorine atom.

Examples of the aryl group having a group selected from Group G6 include a 4-chlorophenyl group, a 4-methoxyphenyl group and a 2,6-dichlorophenyl group.

In the formulae (2-1) and (2-2), R⁵ may be linked to R⁴ to form a divalent hydrocarbon group optionally having a substituent. Examples of the divalent hydrocarbon group include polymethylene groups such as an ethylene group, a trimethylene group and a tetramethylene group; a vinylene group, a propane-1,2-diyl group, a propene-1,2-diyl group, a butane-1,2-diyl group, a 2-butene-1,2-diyl group, a cyclopentane-1,2-diyl group, a cyclohexane-1,2-diyl group and an o-phenylene group.

Examples of the substituent which the divalent hydrocarbon group may have include a group selected from the above Group G2.

In the formula (2-1), examples of the alkyl group for R⁸ include linear or branched C₁-C₆ alkyl groups such as a methyl group, an ethyl group, a propyl group, an isopropyl group, a butyl group, an isobutyl group, a sec-butyl group, a pentyl group and a hexyl group.

The compound (2-1) is preferably a compound represented by the formula (2-3) (hereinafter sometimes referred to as “compound (2-3)”) or a compound represented by the formula (2-4) (hereinafter sometimes referred to as “compound (2-4)”), more preferably the compound (2-3). The compound (2-2) is preferably a compound represented by the formula (2-5) (hereinafter sometimes referred to as “compound (2-5)”) or a compound represented by the formula (2-6) (hereinafter sometimes referred to as “compound (2-6)”), more preferably the compound (2-5).

Namely, the present reaction is preferably carried out by mixing an alcohol, carbon dioxide, an aldehyde and at least one compound selected from the group consisting of the compounds (2-3), (2-4), (2-5) and (2-6). Also, the present reaction is preferably carried out in the presence of at least one compound selected from the group consisting of the compounds (2-3), (2-4), (2-5) and (2-6).

Hereinafter, the compounds (2-3), (2-4), (2-5) and (2-6) will be described.

In the formulae (2-3), (2-4), (2-5) and (2-6), R² has the same meaning as R² in the formulae (2-1) and (2-2), and Y has the same meaning as Y in the formulae (2-1) and (2-2). When Y is a group of —N(R⁵)—, R⁵ has the same meaning as R⁵ in the formulae (2-1) and (2-2).

In the formulae (2-3) and (2-4), R⁸ has the same meaning as R⁸ in the formula (2-1).

In the formulae (2-3), (2-4), (2-5) and (2-6), Y is preferably a group of —N(R⁵)—.

In the formulae (2-3), (2-4), (2-5) and (2-6), it is preferred that at least one of R² and R⁵ is a bulky group.

More preferably, R² and R⁵ are both a bulky group. R² and

R⁵ may be the same or different groups.

Examples of the bulky group for R² and R⁵ include C₄-C₁₂ tertiary alkyl groups such as a tert-butyl group and a tert-pentyl group; C₃-C₁₀ cycloalkyl groups such as a cyclopropyl group, a 2,2-dimethylcyclopropyl group, a cyclopentyl group, a cyclohexyl group, a menthyl group and an adamantyl group; a phenyl group having substituents at least 2 and 6 positions (2,6-disubstituted phenyl group) such as a 2,6-dimethylphenyl group, a 2,6-dichlorophenyl group, a 2,4,6-trimethylphenyl group and a 2,6-diisopropylphenyl group; and a naphthyl group having a C₁-C₁₀ alkyl group at 2 position such as 2-methylnaphthyl group.

Examples of the substituent in the 2,6-disubstituted phenyl group include a C₁-C₁₂ alkyl group and a halogen atom.

The bulky group is preferably a tert-butyl group, a tert-pentyl group, a cyclohexyl group, an adamantyl group or a 2,6-disubstituted phenyl group, more preferably a 2,6-disubstituted phenyl group, and still more preferably a 2,6-diisopropylphenyl group.

Examples of the alkyl group in the “alkyl group optionally having a substituent” for R⁶ in the formulae (2-3) and (2-5) and for R⁷ in the formulae (2-3), (2-4), (2-5) and (2-6) include linear, branched or cyclic C₁-C₁₀ alkyl groups such as a methyl group, an ethyl group, a propyl group, an isopropyl group, a butyl group, an isobutyl group, a sec-butyl group, a tert-butyl group, a pentyl group, a decyl group, a cyclopropyl group, 2,2-dimethylcyclopropyl group, a cyclopentyl group, a cyclohexyl group and a menthyl group.

Examples of the substituent which the alkyl group may have include a group selected from the above Group G4.

Examples of the alkyl group having a group selected from Group G4 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

Examples of the aryl group in the “aryl group optionally having a substituent” for R⁶ in the formulae (2-3) and (2-5) and for R⁷ in the formulae (2-3), (2-4), (2-5) and (2-6) include C₆-C₁₀ aryl groups such as a phenyl group, a 2-methylphenyl group, a 4-methylphenyl group, a 1-naphthyl group and a 2-naphthyl group.

Examples of the substituent which the aryl group may have include a group selected from the above Group G2.

Examples of the aryl group having a group selected from Group G2 include a 4-chlorophenyl group and a 4-methoxyphenyl group.

In the formulae (2-3) and (2-5), R⁶ and R⁷ may be taken together with the carbon atoms to which they are attached to form a ring. Examples of the ring include a cyclopentane ring, a cyclohexane ring and a benzene ring.

In the formulae (2-3) and (2-5), it is preferred that R⁶ and R⁷ each independently represents a hydrogen atom or an alkyl group optionally having a substituent. More preferably, R⁶ and R⁷ are both a hydrogen atom.

In the formulae (2-3) and (2-5), preferably represents a single bound.

Examples of the compound (2-3) include a 2-methoxy-1,3-di-tert-butylimidazolidine, 2-ethoxy-1,3-di-tert-butylimidazolidine, 2-n-propoxy-1,3-di-tert-butylimidazolidine, 2-methoxy-1,3-dicyclohexylimidazolidine, 2-ethoxy-1,3-dicyclohexylimidazolidine, 2-propoxy-1,3-dicyclohexylimidazolidine, 2-methoxy-1,3-diadamantylimidazolidine, 2-methoxy-1,3-diphenylimidazolidine, 2-methoxy-1,3-bis[(2,6-diisopropyl)phenyl]imidazolidine, 2-methoxy-1,3-bis[(2,4,6-trimethyl)phenyl]imidazolidine, 2-ethoxy-1,3-bis[(2,6-diisopropyl)phenyl]imidazolidine, 2-ethoxy-1,3-bis[(2,4,6-trimethyl)phenyl]imidazolidine, 2-propoxy-1,3-bis[(2,6-diisopropyl)phenyl]imidazolidine, 2-propoxy-1,3-bis[(2,4,6-trimethyl)phenyl]imidazolidine, 2-butoxy-1,3-bis[(2,6-diisopropyl)phenyl]imidazolidine, 2-butoxy-1,3-bis[(2,4,6-trimethyl)phenyl]imidazolidine, 2-isopropoxy-1,3-bis[(2,6-diisopropyl)phenyl]imidazolidine, 2-isopropoxy-1,3-bis[(2,4,6-trimethyl)phenyl]imidazolidine, 2-methoxy-4,5-dimethyl-1,3-bis[(2,6-diisopropyl)phenyl]-imidazolidine, 2-methoxy-4,5-dimethyl-1,3-bis[(2,4,6-trimethyl)phenyl]-imidazolidine, 2-ethoxy-4,5-dimethyl-1,3-bis[(2,6-diisopropyl)phenyl]-imidazolidine, 2-ethoxy-4,5-dimethyl-1,3-bis[(2,4,6-trimethyl)phenyl]-imidazolidine, 2-methoxy-4,5-dichloro-1,3-bis[(2,6-diisopropyl)phenyl]-imidazolidine, 2-methoxy-4,5-diphenyl-1,3-bis[(2,4,6-trimethyl)phenyl]-imidazolidine, 2-methoxy-4,5-difluoro-1,3-bis[(2,6-diisopropyl)phenyl]-imidazolidine, 2-methoxy-4-methyl-1,3-bis[(2,4,6-trimethyl)phenyl]-imidazolidine, 2-methoxy-1,3-bis[(2,6-dichloro)phenyl]imidazolidine, 2-methoxy-1-tert-butyl-3-phenylimidazolidine, 2-methoxy-1-cyclohexyl-3-[(2,6-diisopropyl)phenyl]-imidazolidine, 2-methoxy-1-phenyl-3-[(2,4,6-trimethyl)phenyl]imidazolidine, 2-ethoxy-1-tert-butyl-3-[(2,6-diisopropyl)phenyl]-imidazolidine, and 2-ethoxy-1-tert-butyl-3-[(2,4,6-trimethyl)phenyl]-imidazolidine.

Examples of the compound (2-4) include a 5-methoxy-1,4-dimethyl-1,2,4(5H)-triazoline, and 5-methoxy-1,3,4-triphenyl-1,2,4(5H)-triazoline.

Examples of the compound (2-5) include a 2-carboxy-4,5-dihydro-1,3-di-tert-butylimidazolium, 2-carboxy-4,5-dihydro-1,3-dicyclohexylimidazolium, 2-carboxy-4,5-dihydro-1,3-diadamantylimidazolium, 2-carboxy-4,5-dihydro-1,3-diphenylimidazolium, 2-carboxy-4,5-dihydro-1,3-bis[(2,6-diisopropyl)phenyl]-imidazolium, 2-carboxy-4,5-dihydro-1,3-bis[(2,4,6-trimethyl)phenyl]-imidazolium, 2-carboxy-4,5-dihydro-4,5-dimethyl-1,3-bis[(2,6-diisopropyl)phenyl]imidazolium, 2-carboxy-4,5-dihydro-4,5-dimethyl-1,3-bis[(2,4,6-trimethyl)phenyl]imidazolium, 2-carboxy-4,5-dihydro-4,5-dichloro-1,3-bis[(2,6-diisopropyl)phenyl]imidazolium, 2-carboxy-4,5-dihydro-4,5-diphenyl-1,3-bis[(2,4,6-trimethyl)phenyl]imidazolium, 2-carboxy-4,5-dihydro-4,5-difluoro-1,3-bis[(2,6-diisopropyl)phenyl]imidazolium, 2-carboxy-4,5-dihydro-4-methyl-1,3-bis[(2,4,6-trimethyl)-phenyl]imidazolium, 2-carboxy-4,5-dihydro-1,3-bis[(2,6-dichloro)phenyl]-imidazolium, 2-carboxy-4,5-dihydro-1-tert-butyl-3-phenylimidazolium, 2-carboxy-4,5-dihydro-1-cyclohexyl-3-[(2,6-diisopropyl)-phenyl]imidazolium, 2-carboxy-4,5-dihydro-1-phenyl-3-[(2,4,6-trimethyl)phenyl]-imidazolium, 2-carboxy-4,5-dihydro-1-tert-butyl-3-[(2,6-diisopropyl)-phenyl]imidazolium, and 2-carboxy-4,5-dihydro-1-tert-butyl-3-[(2,4,6-trimethyl)-phenyl]imidazolium.

Examples of the compound (2-6) include a 5-carboxy-1,3, 4-triphenyl-4H, 1,2,4-triazolium.

The compounds (2-1) and (2-2) may be a commercially available product or those prepared by, for example, a method described in J. Am. Chem.Soc., Vol. 127, page 9079 (2005).

The amount of at least one compound selected from the group consisting of the compounds (2-1) and (2-2) to be used is preferably 0.001 mol to 0.5 mol, more preferably 0.01 mol to 0.3 mol, relative to 1 mol of an aldehyde.

The carbon dioxide to be used in the present reaction may be in the form of either a gaseous carbon dioxide, a solid carbon dioxide (i.e., dry ice) or a supercritical carbon dioxide. The gaseous carbon dioxide may be diluted with an inert gas such as nitrogen.

The amount of carbon dioxide to be used is preferably 1 mol or more relative to 1 mol of an aldehyde. The upper limit of the amount is not limited.

The present reaction is preferably carried out in the presence of oxygen. The oxygen to be used in the present reaction may be in the form of either a gaseous oxygen, a gaseous oxygen diluted with an inert gas such as nitrogen, an atmospheric oxygen, or an atmospheric oxygen diluted with an inert gas such as nitrogen.

The amount of oxygen to be used is preferably 1 to 100 mol relative to 1 mol of an aldehyde.

The present reaction may be carried out in the presence of a solvent (with the proviso that alcohol solvents are excluded).

The solvent is not limited as long as it does not inhibit the present reaction, but examples thereof include ether solvens such as tetrahydrofuran, methyl tert-butyl ether, cyclopentyl methyl ether, and diisopropyl ether; ester solvents such as ethyl acetate and butyl acetate;

aromatic solvents such as toluene and chlorobenzene; nitrile solvents such as acetonitrile and propionitrile; and a mixture thereof.

The amount of the solvent to be used is not limited, but practically 100 parts by weight or less relative to 1 part by weight of an aldehyde.

In the present reaction, the order of mixing of the reactants is not limited. In a preferred embodiment, for example, an aldehyde, an alcohol, and at least one compound selected from the group consisting of the compounds (2-1) and (2-2), and optionally a solvent, are mixed, and then carbon dioxide is added to the resultant mixture. The mixing is preferably carried out under an atmosphere of an inert gas such as nitrogen.

The present reaction may be carried out under either reduced pressure, normal pressure or increased pressure, preferably normal pressure or increased pressure.

The reaction temperature for the present reaction may vary depending on the kind and amount of at least one compound selected from the group consisting of the compounds (2-1) and (2-2), preferably −20° C. to 150° C., more preferably 0° C. to 100° C. When the reaction temperature is less than −20° C., the reaction rate of the present reaction tends to become lower. When the reaction temperature exceeds 150° C., the selectivity of the present reaction tends to become lower.

The progress of the present reaction can be monitored by analytical means such as gas chromatography, high-performance liquid chromatography, thin-layer chromatography, nucleic magnetic resonance spectrum analysis, or infrared absorption spectrum analysis.

After completion of the reaction, carbon dioxide used in the present reaction and carbon monoxide generated from carbon dioxide by the present reaction can be easily removed as a gas from the resultant reaction mixture. The desired carboxylic acid ester can be taken out from the resultant reaction mixture by, for example, cooling the mixture, optionally after concentration of the mixture. The resultant carboxylic acid ester can be purified by a purification procedure such as distillation, column chromatography, or crystallization.

Thus obtained carboxylic acid ester is a compound represented by the formula (3) (hereinafter sometimes referred to as “compound (3)”) when the aldehyde is the compound (1) and the alcohol is the compound (4).

When the compound (1) wherein n is 0 in the formula (1) is used as an aldehyde, the compound (3) wherein n is 0 in the formula (3) is obtained. Examples of such compound (3) include methyl benzoate, methyl 4-chlorobenzoate, methyl 2-methylbenzoate, methyl 4-fluorobenzoate, methyl 4-methoxybenzoate, methyl 2-nitrobenzoate, methyl 2,4-dichlorobenzoate, methyl 2-naphthalenecarboxylate, methyl 2-pyridinecarboxylate, methyl acetate, methyl propionate, methyl butanoate, methyl pentanoate, methyl hexanoate, methyl heptanoate, methyl octanoate, methyl cyclohexanoate, methyl 4-(methylthio)butanoate, methyl 3-butenoate, methyl acrylate, methyl 3-phenyl-2-propenoate, ethyl benzoate, ethyl 4-chlorobenzoate, ethyl 2-methyl benzoate, ethyl 4-fluorobenzoate, ethyl 4-methoxybenzoate, ethyl 2-nitrobenzoate, ethyl 2,4-dichlorobenzoate, ethyl 2-naphthalenecarboxylate, ethyl 2-pyridinecarboxylate, ethyl acetate, ethyl propionate, ethyl butanoate, ethyl pentanoate, ethyl hexanoate, ethyl heptanoate, ethyl octanoate, ethyl cyclohexanoate, ethyl 4-(methylthio)butanoate, ethyl 3-butenoate, ethyl acrylate, ethyl 3-phenyl-2-propenoate, benzyl benzoate, benzyl 4-chlorobenzoate, benzyl 2-methylbenzoate, benzyl 4-fluorobenzoate, benzyl 4-methoxybenzoate, benzyl 2-nitrobenzoate, benzyl 2,4-dichlorobenzoate, benzyl 2-naphthalenecarboxylate, benzyl 2-pyridinecarboxylate, benzyl acetate, benzyl propionate, benzyl butanoate, benzyl pentanoate, benzyl hexanoate, benzyl heptanoate, benzyl octanoate, benzyl cyclohexanoate, benzyl 4-(methylthio)butanoate, benzyl 3-butenoate, benzyl acrylate and benzyl 3-phenyl-2-propenoate.

When the compound (1) wherein n is 1 in the formula (1) is used as an aldehyde, the compound (3) wherein n is 1 in the formula (3) is obtained. Examples of such compound (3) include methyl benzoylformate, methyl 4-chlorobenzoylformate, methyl 2-methylbenzoylformate, methyl 4-fluorobenzoylformate, methyl 4-methoxybenzoylformate, methyl 2-nitrobenzoylformate, methyl 2,4-dichlorobenzoylformate, methyl 2-naphthoylformate, methyl α-oxo-2-pyridineacetate, methyl pyruvate, methyl 2-oxobutanoate, methyl 2-oxopentanoate, methyl 3-methyl-2-oxobutanoate, methyl α-oxo-cyclohexaneacetate, methyl 4-(methylthio)-2-oxo-butanoate, methyl 2-oxo-3-butenoate, methyl 2-oxo-4-phenyl-3-butenoate, ethyl benzoylformate, ethyl 4-chlorobenzoylformate, ethyl 2-methylbenzoylformate, ethyl 4-fluorobenzoylformate, ethyl 4-methoxybenzoylformate, ethyl 2-nitrobenzoylformate, ethyl 2,4-dichlorobenzoylformate, ethyl 2-naphthoylformate, ethyl α-oxo-2-pyridineacetate, ethyl pyruvate, ethyl 2-oxobutanoate, ethyl 2-oxopentanoate, ethyl 3-methyl-2-oxobutanoate, ethyl α-oxo-cyclohexaneacetate, ethyl 4-(methylthio)-2-oxo-butanoate, ethyl 2-oxo-3-butenoate, ethyl 2-oxo-4-phenyl-3-butenoate, benzyl benzoylformate, benzyl 4-chlorobenzoylformate, benzyl 2-methylbenzoylformate, benzyl 4-fluorobenzoylformate, benzyl 4-methoxybenzoylformate, benzyl 2-nitrobenzoylformate, benzyl 2,4-dichlorobenzoylformate, benzyl 2-naphthoylformate, benzyl α-oxo-2-pyridineacetate, benzyl pyruvate, benzyl 2-oxobutanoate, benzyl 2-oxopentanoate, benzyl 3-methyl-2-oxobutanoate, benzyl α-oxo-cyclohexaneacetate, benzyl 4-(methylthio)-2-oxo-butanoate, benzyl 2-oxo-3-butenoate and benzyl 2-oxo-4-phenyl-3-butenoate.

### EXAMPLES

Hereinafter, the present invention will be described in more detail by way of Examples.

### Example 1

A 50 ml schrenck tube equipped with a magnetic rotor was charged with methylglyoxal monohydrate (140 mg), 2-methoxy-1,3-bis[(2,6-diisopropyl)phenyl]imidazolidine (50 mg), methanol (1 g) and tetrahydrofuran (5 g) under a nitrogen atmosphere, and the resultant mixture was stirred while maintaining the temperature of the mixture in a water bath at 25° C. Dry ice (1.0 g) was added to the mixture to initiate the reaction, and the mixture was stirred at a room temperature under normal pressure for 8 hours. At 30 minutes and 1 hour following the start of the reaction, dry ice (1 g) was added to the reaction mixture, respectively.

After completion of the reaction, carbon dioxide and the by-product carbon monoxide were removed as a gas from the reaction mixture. Then, the resultant reaction mixture was analyzed with a gas chromatography internal standard method to determine that the yield of methyl pyruvate was 10%. In the reaction mixture after completion of the reaction, 80% of methylglyoxal was remained unreacted.

### Example 2

A 100 ml stainless-steel pressure reaction tube equipped with a magnetic rotor was charged with phenylglyoxal monohydrate (200 mg), 2-methoxy-1,3-bis[(2,6-diisopropyl)phenyl]imidazolidine (30 mg) and methanol (5 g) under a nitrogen atmosphere, and the resultant mixture was cooled in a dry ice bath at −70° C. To the cooled mixture was added dry ice (2 g), and then the pressure reaction tube was sealed. The resultant mixture was reacted by stirring it at 60° C. for 6 hours.

After completion of the reaction, carbon dioxide and the by-product carbon monoxide were removed as a gas from the reaction mixture. Then, the resultant reaction mixture was analyzed with a gas chromatography internal standard method to determine that the yield of methyl benzoylformate was 7%. In the reaction mixture after completion of the reaction, 90% of phenylglyoxal was remained unreacted.

### Example 3

A 100 ml stainless-steel pressure reaction tube equipped with a magnetic rotor was charged with 4-(methylthio)-2-oxo-1- butanal (100 mg), 2-methoxy-1,3-bis[(2,6-diisopropyl)phenyl] imidazolidine (20 mg), methanol (500 mg) and tetrahydrofuran (2 g) under a nitrogen atmosphere, and the resultant mixture was cooled in a dry ice bath at -70° C. To the cooled mixture was added dry ice (2 g), and then the pressure reaction tube was sealed. The resultant mixture was reacted by stirring it at 60° C. for 6 hours.

After completion of the reaction, carbon dioxide and the by-product carbon monoxide were removed as a gas from the reaction mixture. Then, the resultant reaction mixture was analyzed with a gas chromatography internal standard method to determine that the yield of methyl 4-(methylthio)-2-oxo- butanoate was 20%. In the reaction mixture after completion of the reaction, 30% of 4-(methylthio)-2-oxo-1-butanal was remained unreacted.

### Example 4

A 100 ml stainless-steel pressure reaction tube equipped with a magnetic rotor was charged with n-hexyl aldehyde (140 mg), 2-methoxy-1,3-bis[(2,6-diisopropyl)phenyl] imidazolidine (30 mg), methanol (100 mg) and tetrahydrofuran (3 g) under a nitrogen atmosphere, and the resultant mixture was cooled in a dry ice bath at −70° C. To the cooled mixture was added dry ice (2 g), and then the pressure reaction tube was sealed. The resultant mixture was reacted by stirring it at 60° C. for 6 hours.

After completion of the reaction, the resultant reaction mixture was analyzed with a gas chromatography internal standard method to determine that the yield of methyl hexanoate was 14%. In the reaction mixture after completion of the reaction, 80% of n-hexyl aldehyde was remained unreacted.

After completion of the reaction, carbon dioxide gas was released from the pressure reaction tube to return the pressure to normal. The gas was analyzed with a CO concentration meter (gaseous detector XX2200T, Cosmos Electric Co., Ltd.) to determine that the concentration of carbon monoxide in the gas was 78 ppm.

### Example 5

A 100 ml stainless-steel pressure reaction tube equipped with a magnetic rotor was charged with 4-(methylthio)-2-oxo-1-butanal (100 mg), 2-carboxy-4,5-dihydro-1,3-bis[(2,4,6-trimethyl)phenyl]imidazolium (10 mg), methanol (500 mg) and tetrahydrofuran (3 g) under a nitrogen atmosphere, and the resultant mixture was cooled in a dry ice bath at -70° C. To the cooled mixture was added dry ice (2 g), and then the pressure reaction tube was sealed. The resultant mixture was reacted by stirring it at 60° C. for 4 hours.

After completion of the reaction, carbon dioxide and the by-product carbon monoxide were removed as a gas from the reaction mixture. Then, the resultant reaction mixture was analyzed with a gas chromatography internal standard method to determine that the yield of methyl 4-(methylthio)-2-oxo- butanoate was 10%.

### Example 6

A 100 ml stainless-steel pressure reaction tube equipped with a magnetic rotor was charged with n-hexyl aldehyde (140 mg), 5-methoxy-1,3,4-triphenyl-1,2,4(5H)-triazoline (23 mg), methanol (100 mg) and tetrahydrofuran (2 g) under a nitrogen atmosphere, and the resultant mixture was cooled in a dry ice bath at −70° C. To the cooled mixture was added dry ice (2 g), and then the pressure reaction tube was sealed. The resultant mixture was reacted by stirring it at 60° C. for 6 hours.

After completion of the reaction, carbon dioxide and the by-product carbon monoxide were removed as a gas from the reaction mixture. Then, the resultant reaction mixture was analyzed with a gas chromatography internal standard method to determine that the yield of methyl hexanoate was 5%. In the reaction mixture after completion of the reaction, 80% of n-hexyl aldehyde was remained unreacted.

### Example 7

A 100 ml stainless-steel pressure reaction tube equipped with a magnetic rotor was charged withbenzaldehyde (150 mg), 2-methoxy-1,3-bis[(2,6-diisopropyl)phenyl]imidazolidine (30 mg), methanol (140 mg) and tetrahydrofuran (2 g) under a nitrogen atmosphere, and then the resultant mixture was cooled in a dry ice bath at −70° C. To the cooled mixture was added dry ice (2 g), and then the pressure reaction tube was sealed. The resultant mixture was reacted by stirring it at 40° C. for 6 hours.

After completion of the reaction, carbon dioxide and the by-product carbon monoxide were removed as a gas from the reaction mixture. Then, the resultant reaction mixture was analyzed with a gas chromatography internal standard method to determine that the yield of methyl benzoate was 5%. In the reaction mixture after completion of the reaction, 92% of benzaldehyde was remained unreacted.

After completion of the reaction, carbon dioxide gas was released from the pressure reaction tube to return the pressure to normal. The gas was analyzed with a CO concentration meter (gaseous detector XX2200T, Cosmos Electric Co., Ltd.) to determine that the concentration of carbon monoxide in the gas was 98 ppm.

### Example 8

A 100 ml stainless-steel pressure reaction tube equipped with a magnetic rotor was charged with phenylglyoxal monohydrate (130 mg), 2-carboxy-4,5-dihydro-1,3-bis[(2,4,6-trimethyl)phenyl]imidazolium (30 mg), methanol (300 mg) and tetrahydrofuran (3 g) under a nitrogen atmosphere, and the resultant mixture was cooled in a dry ice bath at −70° C. To the cooled mixture was added dry ice (2 g), and then the pressure reaction tube was sealed. The resultant mixture was pressured to 1 MPa with air, and then reacted by stirring it at 60° C. for 3 hours.

After completion of the reaction, air, carbon dioxide and the by-product carbon monoxide were removed as a gas from the reaction mixture. Then, the resultant reaction mixture was analyzed with a gas chromatography internal standard method to determine that the yield of methyl benzoylformate was 31%. In the reaction mixture after completion of the reaction, 35% of phenylglyoxal was remained unreacted.

### INDUSTRIAL APPLICABILITY

Carboxylic acid esters are important compounds as various chemical products. Carboxylic acid esters such as ketocarboxylic acid esters are known to be useful as intermediates in the preparation of amino acids and the like. The present invention is industrially available as a process for producing a carboxylic acid ester.

1. A process for producing a carboxylic acid ester, comprising a step of oxidizing an aldehyde by mixing an alcohol, carbon dioxide, the aldehyde and at least one compound selected from the group consisting of a compound represented by the formula (2-1): 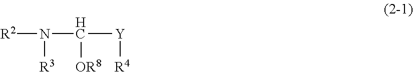
wherein R² represents an alkyl group optionally having a substituent or an aryl group optionally having a substituent;
R³ and R⁴ each independently represents an alkyl group optionally having a substituent or an aryl group optionally having a substituent, or R³ and R⁴ are linked together to form a divalent hydrocarbon group optionally having a substituent or a group of —CH=N— optionally having a substituent;
Y represents a group of —S— or a group of —N(R⁵)—, wherein R⁵ represents an alkyl group optionally having a substituent or an aryl group optionally having a substituent, or R⁵ is linked to R⁴ to form a divalent hydrocarbon group optionally having a substituent; and
R⁸ represents an alkyl group; and
a compound represented by the formula (2-2): 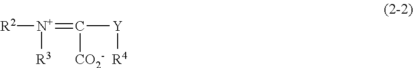
wherein R², R³, R⁴ and Y are as defined above.

2. The process according to claim 1, wherein the process is carried out in the presence of oxygen.

3. The process according to claim 1, wherein the aldehyde is a compound represented by the formula (1): 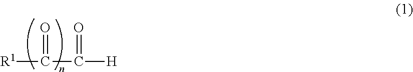
wherein R¹ represents a hydrocarbon group optionally having a substituent or a heteroaryl group optionally having a substituent, and
n represents 0 or 1;
the alcohol is a compound represented by the formula (4):
R⁹—OH (4)
wherein R⁹ represents an alkyl group optionally having a substituent; and
the carboxylic acid ester is a compound represented by the formula (3): 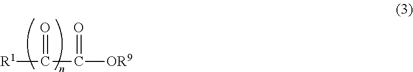
wherein R¹, R⁹ and n are as defined above.

4. The process according to claim 1, wherein the compound represented by the formula (2-1) is a compound represented by the formula (2-3): 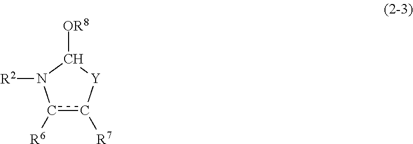
wherein R², R⁸ and Y are as defined above;
R⁶ and R⁷ each independently represents a hydrogen atom, an alkyl group optionally having a substituent or an aryl group optionally having a substituent, or R⁶ and R⁷ are taken together with the carbon atoms to which they are attached to form a ring; and [symbol]
represents a single bound or a double bond; or
a compound represented by the formula (2-4): 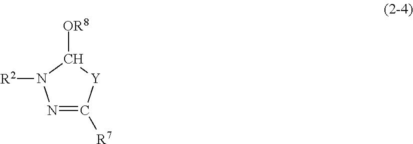
wherein R², R⁷, R⁸ and Y are as defined above; and
the compound represented by the formula (2-2) is a compound represented by the formula (2-5): 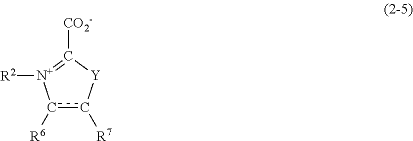
wherein R², R⁶, R⁷ and Y are as defined above; or
a compound represented by the formula (2-6): 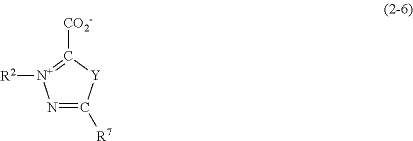
wherein R², R⁷ and Y are as defined above.

5. The process according to claim 4, wherein in the formulae (2-3), (2-4), (2-5) and (2-6), Y is a group of —N(R⁵)—; and R² and R⁵ each is independently a phenyl group optionally having a substituent.

6. A process for producing a carboxylic acid ester, comprising a step of reacting an aldehyde, an alcohol and carbon dioxide in the presence of at least one compound selected from the group consisting of a compound represented by the formula (2-1): 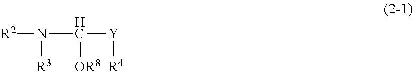
wherein R² represents an alkyl group optionally having a substituent or an aryl group optionally having a substituent; R³ and R⁴ each independently represents an alkyl group optionally having a substituent or an aryl group optionally having a substituent, or R³ and R⁴ are linked together to form a divalent hydrocarbon group optionally having a substituent or a group of —CH═N— optionally having a substituent;
Y represents a group of —S— or a group of —N(R⁵)—, wherein R⁵ represents an alkyl group optionally having a substituent or an aryl group optionally having a substituent, or R⁵ is linked to R⁴ to form a divalent hydrocarbon group optionally having a substituent; and
R⁸ represents an alkyl group; and
a compound represented by the formula (2-2): 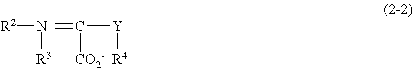
wherein R², R³, R⁴ and Y are as defined above.

7. The process according to claim 6, wherein the process is carried out in the presence of oxygen.

8. The process according to claim 6, wherein the aldehyde is a compound represented by the formula (1): 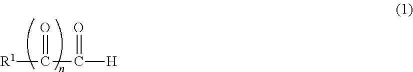
wherein R¹ represents a hydrocarbon group optionally having a substituent or a heteroaryl group optionally having a substituent; and
n represents 0 or 1;
the alcohol is a compound represented by the formula (4):
R⁹—OH (4)
wherein R⁹ represents an alkyl group optionally having a substituent; and
the carboxylic acid ester is a compound represented by the formula (3): 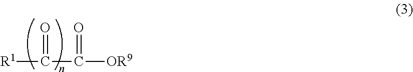
wherein R¹, R⁹ and n are as defined above.

9. The process according to claim 6, 7 or 8, wherein the compound represented by the formula (2-1) is a compound represented by the formula (2-3): 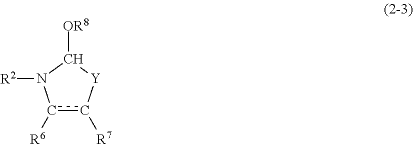
wherein R², R⁸ and Y are as defined above;
R⁶ and R⁷ each independently represents a hydrogen atom, an alkyl group optionally having a substituent or an aryl group optionally having a substituent, or R⁶ and R⁷ are taken together with the carbon atoms to which they are attached to form a ring; and [symbol]
represents a single bound or a double bond; or
a compound represented by the formula (2-4): 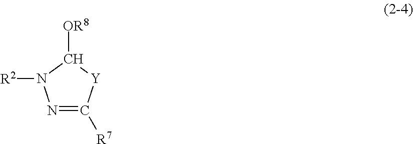
wherein R², R⁷, R⁸ and Y are as defined above; and
the compound represented by the formula (2-2) is a compound represented by the formula (2-5): 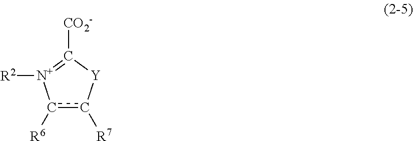
wherein R², R⁶, R⁷ and Y are as defined above; or
a compound represented by the formula (2-6): 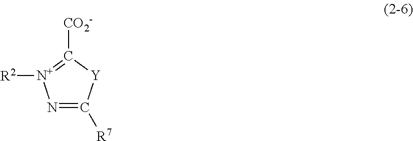
wherein R², R⁷ and Y are as defined above.

10. The process according to claim 9, wherein in the formulae (2-3), (2-4), (2-5) and (2-6), Y is a group of —N(R⁵)—; and R² and R⁵ each is independently a phenyl group optionally having a substituent.
